SEQUENCE LISTING (12 sequences)

SEQ ID NO: 1 - protein, 31 aa, Homo sapiens
  VARIANT (31)...(31): Valine or Alanine
SETTTGTSSNSSQSTSNSGLAPNPTNATTKX

SEQ ID NO: 2 - protein, 30 aa, Homo sapiens
SETTTGTSSNSSQSTSNSGLAPNPTNATTK

SEQ ID NO: 3 - protein, 27 aa, Mus musculus
NQTSVAPFPGNQNISASPNPTNATTRG

SEQ ID NO: 4 - protein, 26 aa, Homo sapiens
MGRAMVARLGLGLLLLALLLPTQIYS

SEQ ID NO: 5 - protein, 287 aa, Artificial
  Fusion protein
MGRAMVARLGLGLLLLALLLPTQIYSSETTTGTSSNSSQSTSNSGLAPNPTNATTKPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 261 aa, Artificial
  Fusion protein
SETTTGTSSNSSQSTSNSGLAPNPTNATTKPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 231 aa, Homo sapiens
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 288 aa, Artificial
  Fusion protein
MGRAMVARLGLGLLLLALLLPTQIYSSETTTGTSSNSSQSTSNSGLAPNPTNATTKVPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 288 aa, Artificial
  Fusion protein
MGRAMVARLGLGLLLLALLLPTQIYSSETTTGTSSNSSQSTSNSGLAPNPTNATTKAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 29 aa, Macaca fascicularis
TVTTSAPLSSNSPQNTSTTPNPANTTTKA

SEQ ID NO: 11 - protein, 262 aa, Artificial
  Fusion protein
SETTTGTSSNSSQSTSNSGLAPNPTNATTKVPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 262 aa, Artificial
  Fusion protein
SETTTGTSSNSSQSTSNSGLAPNPTNATTKAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2023/0108492 A1
(43) Pub. Date: Apr. 6, 2023

(54) METHODS OF USE OF SOLUBLE CD24 FOR TREATING VIRAL PNEUMONIA

(71) Applicants: OncoImmune, Inc., Wilmington, DE (US); Kunming Institute of Zoology, Chinese Academy of Sciences, Kunming (CN)

(72) Inventors: Yang Liu, Potomac, MD (US); Pan Zheng, Potomac, MD (US); Martin Devenport, Gaithersburg, MD (US); Renrong Tian, Kunming (CN); Yongtang Zheng, Kunming (CN)

(21) Appl. No.: 17/796,307

(22) PCT Filed: Feb. 10, 2021
(86) PCT No.: PCT/CN21/76548

§ 371 Date: Jul. 29, 2022

Related U.S. Application Data
(60) Provisional application No. 63/007,751, filed on Apr. 9, 2020.

(30) Foreign Application Priority Data
Feb. 10, 2020 (CN) 202010086132.6

(51) Int. Cl. C07K 14/705; A61P 31/12; C12N 15/86

(52) U.S. Cl. CPC C07K 14/70596; A61P 31/12; C12N 15/86; C07K 2319/30

(57) ABSTRACT

Provided is the use of a CD24 protein for treating viral pneumonia.

### FIELD OF THE INVENTION

The present invention relates to compositions and methods for treating or preventing viral pneumonia.

### BACKGROUND OF THE INVENTION

Viral pneumonia is a major cause of mortality in both systemic and respiratory infections, with antivirals being the primary therapeutic options. However, since pneumonia may persist after viral clearance, other non-antiviral therapeutic options should be considered. An intriguing possibility is viral cytopathic effects may result in release of danger-associated molecular patterns (also referred to as damage-associated molecular patterns or DAMPS) that cause a self-propagating inflammatory response with lasting lung damage. Accordingly, it is of interest to test immunotherapeutics that target DAMP-induced inflammation for the treatment of viral pneumonia.

### SUMMARY OF THE INVENTION

Provided herein is a method of treating or preventing a viral pneumonia in a subject in need thereof. The method may comprise administering an effective amount of a CD24 protein to the subject. Also provided herein is use of the CD24 protein in the manufacture of a medicament for treating a viral pneumonia. In an embodiment of the present invention, the pneumonia is caused by one or more of an influenza virus, a parainfluenza virus, a respiratory syncytial virus, or a human immunodeficiency virus. In another embodiment of the present invention, the pneumonia is caused by a secondary infection, which may be bacterial, following a viral infection.

In one embodiment of the disclosed method the CD24 protein comprises a mature CD24 polypeptide or variant thereof. In another embodiment the CD24 protein comprises a mature human CD24 polypeptide or variant thereof. In another embodiment the mature human CD24 polypeptide is a polypeptide of SEQ ID NO. 1 or SEQ ID NO. 2. In another embodiment the mature human CD24 polypeptide is a polypeptide of SEQ ID NO. 1. In another embodiment the mature human CD24 polypeptide is a polypeptide of SEQ ID NO. 1, wherein the C-terminal amino acid is valine. In another embodiment the mature human CD24 polypeptide is a polypeptide of SEQ ID NO. 1, wherein the C-terminal amino acid is alanine. In another embodiment the mature human CD24 polypeptide is a polypeptide of SEQ ID NO. 2.

In another embodiment of the disclosed method the CD24 protein comprises a protein tag. An aspect of this embodiment is realized when the protein tag is fused to the N-terminus or C-terminus of a mature CD24 polypeptide or variant thereof. Another aspect of this embodiment is realized when the protein tag comprises a portion of a mammalian immunoglobulin (Ig) protein. Another aspect of this embodiment is realized when the portion of the mammalian Ig protein is a Fc region. In another embodiment of the disclosed method the CD24 protein comprises a protein tag fused to the N-terminus or C-terminus of the mature human CD24 polypeptide or variant thereof. Another aspect of this embodiment is realized when the protein tag comprises a portion of a human immunoglobulin (Ig) protein. Another aspect of this embodiment is realized when the portion of the human Ig protein is a Fc region. Another aspect of this embodiment is realized when the Fc region comprises a hinge region and CH2 and CH3 domains of a human Ig protein selected from the group consisting of IgG1, IgG2, IgG3, IgG4, and IgA. Another aspect of this embodiment is realized when the Fc region comprises a hinge region and CH2, CH3 and CH4 domains of IgM. Still another aspect of this embodiment is realized when the amino acid sequence of the CD24 protein comprises a sequence set forth in SEQ ID NO: 6, 11, or 12. Another aspect of this embodiment is realized when the amino acid sequence of the CD24 protein comprises a sequence set forth in SEQ ID NO: 6. Another aspect of this embodiment is realized when the amino acid sequence of the CD24 protein comprises a sequence set forth in SEQ ID NO: 11. Another aspect of this embodiment is realized when the amino acid sequence of the CD24 protein comprises a sequence set forth in SEQ ID NO: 12. A further aspect of this embodiment is realized when the amino acid sequence of the CD24 protein consists of a sequence set forth in SEQ ID NO: 6, 11, or 12. Another aspect of this embodiment is realized when the amino acid sequence of the CD24 protein consists of a sequence set forth in SEQ ID NO: 6. Another aspect of this embodiment is realized when the amino acid sequence of the CD24 protein consists of a sequence set forth in SEQ ID NO: 11. Another aspect of this embodiment is realized when the amino acid sequence of the CD24 protein consists a sequence set forth in SEQ ID NO: 12.

As illustrated herein, the subject may have or be diagnosed with viral pneumonia. In another embodiment of the invention, the CD24 protein is administered intravenously, over a period of 30 minutes to 8 hours. In one class of this embodiment, the CD24 protein is administered intravenously over a period of 30, 40, 50, 60, 70, 80, 90, 100, 110, or 120 minutes, or up to 3, 4, 5, 6, 7 or 8 hours. In another class of this embodiment, the CD24 protein is administered intravenously over a period of about 60 minutes. In a further embodiment of the present invention, the CD24 protein is diluted in an appropriate vehicle. In one aspect of this embodiment, the vehicle is phosphate-buffered saline (PBS)or normal saline (NS). In a class of this embodiment, the CD24 protein is diluted in an appropriate vehicle, such as PBS. In yet another class, the dose of the CD24 protein is diluted to 100 mL, in an appropriate vehicle, such as PBS. In certain embodiments of the present invention, the CD24 protein is administered with a second treatment, such as treatments that are the standard of care treatment for viral pneumonia. The second treatment may be one or more of oxygen therapy, mechanical ventilation, extracorporeal membrane oxygenation, non-invasive ventilation, a high flow oxygen device, remdesivir, a corticosteroid, and an immune modulator.

Administering the CD24 protein as part of the disclosed method may result in one or more of: a reduced risk of death; a reduced duration of treatment with mechanical ventilation, a reduced duration of treatment with extracorporeal membrane oxygenation, a reduced duration of treatment with non-invasive ventilation, a reduced duration of treatment with pressors, a reduced duration of treatment with a high flow oxygen device, a decreased rate of disease progression, an increased time to clinical relapse, a reduced duration of supplemental oxygen, a reduced time of hospital stay, a reduced absolute lymphocyte count, reduced levels of one or more markers of inflammation compared with viral pneumonia patients who were treated with placebo or standard of care treatment,

In one embodiment of the present invention, the CD24 protein administered as part of the disclosed method is soluble. In another embodiment of the disclosed method the CD24 protein is glycosylated.

In one embodiment of the present invention, the CD24 protein administered by the method described herein is produced using a eukaryotic expression system. In one aspect of this embodiment, the expression system comprises a vector contained in a Chinese Hamster Ovary cell line or a replication-defective retroviral vector. In a class of this embodiment, the replication-defective retroviral vector is stably integrated into the genome of the eukaryotic cell.

### BRIEF DESCRIPTION OF THE DRAWINGS

FIGS. 1A-C. FIG. 1A shows the amino acid composition of the full-length version of a CD24 fusion protein (CD24Fc) with a signal peptide (also referred to herein as CD24Ig) (SEQ ID NO: 5). The underlined 26 amino acids (SEQ ID NO: 4) represent the signal peptide, which is cleaved off during secretion from a cell expressing the protein and thus missing from the processed version of the CD24 protein as represented by SEQ ID NO: 6. The bold portion of the sequence (SEQ ID NO: 2) is the extracellular domain of the processed CD24 protein, The last (C-terminal) amino acid (A or V) that is ordinarily present in a mature CD24 protein has been deleted from the fusion protein construct to reduce immunogenicity. The non-underlined, non-bold letters are the sequence of IgG1 Fc, including the hinge region and CH2 and CH3 domains (SEQ ID NO: 7). FIG. 1B shows the sequence of CD24^{V}Fc (SEQ ID NO: 8), in which the mature human CD24 protein (bold) is the valine polymorphic variant of SEQ ID NO: 1. FIG. 1C shows the sequence of CD24^{A}Fc (SEQ ID NO: 9), in which the mature human CD24 protein (bold) is the alanine polymorphic variant of SEQ ID NO: 1. The various parts of the fusion protein in FIGS. 1B and 1C are marked as in FIG. 1A and the variant valine/alanine amino acid is double underlined.

FIG. 2 shows amino acid sequence variations between mature CD24 polypeptides from mouse (SEQ ID NO: 3) and human (SEQ ID NOs: 1 and 2). The potential O-glycosylation sites are bolded, and the N-glycosylation sites are underlined.

FIGS. 3A-C. WinNonlin compartmental modeling analysis of pharmacokinetics of CD24IgG1 (CD24Fc). The opened circles represent the average of 3 mice, and the line is the predicted pharmacokinetic curve. FIG. 3A. Intravenous (i.v.) injection of 1 mg CD24IgG1 (CD24Fc). FIG. 3B. Subcutaneous (s.c.) injection of 1 mg CD24IgG1 (CD24Fc). FIG. 3C. Comparison of the total amounts of antibody in the blood as measured by areas under curve (AUC), half-life and maximal blood concentration. Note that overall, the AUC and Cmax of the s.c. injection are about 80% of i.v. injection, although the difference is not statistically significant.

FIGS. 4A-B. CD24-Siglec G (10) interaction discriminates between pathogen associated molecular pattern (PAMP) and DAMP. FIG. 4A. Host response to PAMP was unaffected by CD24-Siglec G(10) interaction. FIG. 4B. CD24-Siglec G (10) interaction represses host response to DAMP, possibly through the Siglec G/10-associated SHP-1.

FIGS. 5A-C. CD24Fc binds to Siglec 10 and HMGB1 and activates Siglec G, the mouse homologue of human Siglec 10. FIG. 5A. Affinity measurement of the CD24Fc-Siglec 10 interaction. FIG. 5B. CD24Fc specifically interacts with HMGB-1 in a cation-dependent manner. CD24Fc was incubated with HMGB1 in 0.1 mM of CaCl₂ and MgCl₂, in the presence or absence of the cation chelator EDTA. CD24Fc is pulled down with protein G-beads, and the amounts of HMGB1, CD24Fc or control Fc is determined by Western blot. FIG. 5C. CD24Fc activates mouse Siglec G by inducing Tyrosine phosphorylation (middle panel) and association with SHP-1 (upper panel). The amounts of Siglec G are shown in the lower panel. CD24^{-/-} spleen cells were stimulated with 1 μg/ml of CD24Fc, control Fc or vehicle (phosphate buffer saline, PBS) control for 30 minutes. Siglec G was then immunoprecipitated and probed with anti-phospho-tyrosine or anti-SHP-1.

FIGS. 6A-B. CD24Fc inhibits production of TNF-α (FIG. 6A) and IFN-γ (FIG. 6B) by anti-CD3 activated human T cells. The human peripheral blood mononuclear lymphocytes (PBML) were stimulated with anti-CD3 for 4 days in the presence or absence of CD24Fc and the amounts of IFN-γ and TNF-α released in the supernatant of cell culture were measured by ELISA. Data shown are means of triplicate. Error bar, SEM.

FIGS. 7A-B. CD24 inhibits inflammatory cytokine production by human macrophages. FIG. 7A. ShRNA silencing of CD24 leads to spontaneous production of TNF-α, IL-1β, and IL-6. THP1 cells were transduced with lentiviral vectors encoding either scrambled or two independent CD24 shRNA molecules. The transduced cells were differentiated into macrophages by culturing for 4 days with phorbol 12-myristate 13-acetate (PMA) (15 ng/ml). After washing away PMA and non-adherent cells, the cells were cultured for another 24 hours for measurement of inflammatory cytokines, by cytokine beads array. FIG. 7B. As in FIG. 7A, except that the given concentration of CD24Fc or control IgG Fc was added to macrophages in the last 24 hours. Data shown in FIG. 7A are means and S.D. from three independent experiments, while those in FIG. 7B are representative of at least 3 independent experiments.

FIGS. 8A-B. CD24Fc protects SIV-infected Chinese rhesus monkeys against viral pneumonia. FIG. 8A. Experimental protocol. The timeline of the study activities is shown on top. The rhesus monkeys were randomized into two weeks at 8 weeks after infection. Lung tissues were harvested either after premature death at autopsy or necropsy at the termination of the study. Dark lines depict survival of individual monkey receiving normal saline, while the gray lines depicts survival of monkeys receiving CD24Fc. FIG. 8B. Representative images of pathology lesions in individual animals receiving normal saline (left) or CD24Fc. Size bar: 50 μm.

FIG. 9. Hyaline formation in alveoli of lung of normal saline-treated animal number 06047. Four representative lesions are indicated with arrows.

FIG. 10. Hyaline formation in alveoli (gray arrows toward left side) and desquamation of pneumocytes (black arrow toward right side) in the lung section of normal saline-treated animal number 07099.

FIG. 11. Giant cell formation in lung section of normal saline-treated animal number 06047.

### DETAILED DESCRIPTION

The inventors have discovered that a soluble form of CD24 protein is highly effective for treating a viral pneumonia. An aspect of this invention is realized when the CD24 protein is a mature human CD24 polypeptide (SEQ ID NO. 1, or a variant of the mature human CD24 polypeptide wherein the last (C-terminal) amino acid (A or V) of the mature CD24 polypeptide has been deleted. Mature human CD24 protein as used herein, is also sometimes referred to as mature human CD24 polypeptide. Another aspect of the invention is realized when said mature human CD24 polyptide or variant thereof is fused to an IgG1 Fc domain. The effect may be mediated through DAMPs. Pattern recognition is involved in inflammatory response triggered by both pathogen-associated and tissue damage-associated molecular patterns, respectively called PAMPs and DAMPs. The inventors have realized that recent studies have demonstrated that an exacerbated host response to DAMPs may play a part in the pathogenesis of inflammatory and autoimmune disease (Chen, G. Y., et al., CD24 and Siglec-10 selectively repress tissue damage induced immune responses, Science, Vol. 323, pp. 1722-1725 (2009); Liu, Y., et al., CD24-Siglec G/10 discriminates danger-from pathogen-associated molecular patterns, Trends Immunol, Vol. 30, pp. 557-561 (2009); Fang, X., et al., CD24: from A to Z. Cell Mol Immunol, Vol. 7, pp. 100-103 (2010)). DAMPs were found to promote the production of inflammatory cytokines and autoimmune diseases in animal models, and inhibitors of DAMPs such as HMGB1 and HSP90 were consequently found to ameliorate rheumatoid arthritis (RA). TLRs, RAGE-R, DNGR (encoded by Clec9A), and Mincle have been shown to be receptors responsible for mediating inflammation initiated by a variety of DAMPs.

The inventors' recent work demonstrated that CD24-Siglec G interactions discriminate innate immunity to DAMPs from PAMPs. Siglec proteins are membrane-associated immunoglobulin (Ig) superfamily members that recognize a variety of sialic acid-containing structures. Most Siglecs have an intra-cellular immune-tyrosine inhibitory motif (ITIM) that associates with SHP-1, -2 and Cbl-b to control key regulators of inflammatory responses. The inventors have reported CD24 as the first natural ligand for a Siglec, Siglec G in mouse and Siglec 10 in human . Siglec G interacts with sialylated CD24 to suppress the TLR-mediated host response to DAMPs, such as HMGB1, via a SHP-1/2 signaling mechanism.

Human CD24 is a small glycosylphosphatidylinositol (GPI)-anchored molecule encoded by an open-reading frame of 240 base pairs in the CD24 gene. Of the 80 amino acids, the first first 26 amino acids at the NH₂-terminus of the protein constitute the signal peptide, while the last 23 amino acids at the COOH-terminus serve as a signal for cleavage to allow for the attachment of the GPI tail. As a result, the mature human CD24 polypeptide has only 31 amino acids, which also represents the extracellular domain of the human CD24 protein.

One of the 31 amino acids is polymorphic among the human population. A cytosine (C) to thymine (T) nucleotide transition at nucleotide 170 of the CD24 gene open-reading frame results in the amino acid substitution of alanine (A) with valine (V) [at amino acid residue 31 of the mature CD24 protein]. Since this residue is in the immediate N-terminal to the cleavage site, and since the replacement is nonconservative, these two alleles may be expressed at different efficiencies on the cell surface. Indeed, transfection studies with cDNA demonstrated that the CD24^{v} allele is more efficiently expressed on the cell surface. Consistent with this, CD24^{v/v} PBL expressed higher levels of CD24, especially on T cells. Human CD24 is a small GPI-anchored protein encoded by an open-reading frame of 240 nucleotide base pairs in the CD24 gene. Of the 80 amino acids, the first 26 amino acids at the NH₂-terminus constitute the signal peptide, while the last 23 amino acids at the COOH-terminus serve as a signal for cleavage to allow for the attachment of the GPI tail. As a result, the mature human CD24 protein has only 31 amino acids, which also represents the extracellular domain of the CD24protein. One of the 31 amino acids is polymorphic among the human population. A cytosine (C) to thymine (T) nucleotide transition at nucleotide 170 of the CD24 gene open-reading frame results in the amino acid substitution of alanine (A) with valine (V). Since this amino acid residue is positioned immediately N-terminal to the GPI signal cleavage site, and since the replacement is nonconservative, these two alleles may be expressed at different efficiencies on the cell surface. Indeed, transfection studies with cDNA demonstrated that the CD24^{v} allele (containing the valine residue) is more efficiently expressed on the cell surface. Consistent with this, CD24^{v/v} PBL expressed higher levels of CD24, especially on T cells.

The inventors have demonstrated that CD24 negatively regulates host response to cellular DAMPs that are released as a result of tissue or organ damage, and at least two overlapping mechanisms may explain this activity (Chen, G. Y., et al., CD24 and Siglec-10 selectively repress tissue damage-induced immune responses, Science, Vol. 323, pp. 1722-1725 (2009)). First, CD24 binds to several DAMPs, including HSP70, HSP90, HMGB1 and nucleolin and represses host response to these DAMPs. To do this, it is presumed that CD24 may trap the inflammatory stimuli to prevent interaction with their receptors, TLR or RAGE. Second, using an acetaminophen-induced mouse model of liver necrosis and ensuring inflammation, the inventors demonstrated that through interaction with its receptor, Siglec G, CD24 provides a powerful negative regulation for host response to tissue injuries. To achieve this activity, CD24 may bind and stimulate signaling by Siglec G wherein Siglec G-associated SHP1 triggers the negative regulation. Both mechanisms may act in concert as mice with targeted mutation of either gene mounted much stronger inflammatory response. In fact, dendritic cells cultured from bone marrow from either CD24^{-/-} or Siglec G^{-/-} mice produced higher levels of inflammatory cytokines when stimulated with either HMGB1, HSP70, or HSP90. CD24 protein appears to be the only inhibitory DAMP receptor capable of shutting down inflammation triggered by DAMPs and no drug is currently available that specifically targets host inflammatory response to tissue injuries. Furthermore, the inventors have demonstrated the ability of exogenous soluble CD24 protein to alleviate DAMP-mediated autoimmune disease using mouse models of RA, multiple sclerosis (MS) and graft versus host disease (GvHD).

As CD24Fc is an agonist of Siglecs and can fortify the CD24-Siglec innate immune checkpoint, the inventors evaluated whether CD24Fc could improve the lung pathology of SIV-infected rhesus monkeys that received either placebo (Normal saline, NS) or CD24Fc. In particular, they demonstrated that CD24-Siglec pathway can protect against destructive inflammation triggered by cell death. Since death of pneumocytes is a prominent feature of virally induced pneumonia, soluble forms of CD24Fc can be used as a treatment for viral pneumonia, including those caused by HIV, influenza, parainfluenza, and respiratory syncytial virus.

In an embodiment of the invention, the CD24 protein administered by the methods described herein may comprise a mature human CD24 polypeptide, which constitutes the extracellular domain (ECD) of the CD24 protein, or a variant thereof. The mature human CD24 polypeptide or variant thereof is represented by SEQ ID NO: 1 or 2. The CD24 protein may comprise a protein tag, which may be fused at the N-terminus or C-terminus of the CD24 protein. The protein tag may comprise a portion of a mammalian immunoglobulin (Ig) protein, and the portion may be a Fc region. The Ig protein may be human. The Fc region may comprise a hinge region and CH2 and CH3 domains of a human Ig protein selected from the group consisting of IgG1, IgG2, IgG3, IgG4, and IgA. The Fc region may comprise a hinge region and CH2, CH3, and CH4 domains of IgM. The amino acid sequence of the CD24 protein may comprise or consist of a sequence set forth in SEQ ID NO: 6, 11, or 12.

### 1. Definitions.

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

A “peptide” or “polypeptide” is a linked sequence of amino acids and may be natural, synthetic, or a modification or combination of natural and synthetic.

“Substantially identical” may mean that a first and second amino acid sequence are at least 60%, 65%, 70%, 75%, 80%, 85%, 90%, 95%, 96%, 97%, 98%,or 99% over a region of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, or 300 amino acids.

“Treatment” or “treating,” when referring to protection of an animal from a disease, means preventing, suppressing, repressing, or completely eliminating the disease and/or infection. Preventing the disease involves administering a composition of the present invention to an animal prior to onset of the disease. Suppressing the disease involves administering a composition of the present invention to an animal after induction of the disease but before its clinical appearance. Repressing the disease involves administering a composition of the present invention to an animal after clinical appearance of the disease.

A “variant” may mean a peptide or polypeptide that differs in amino acid sequence by the insertion, deletion, or conservative substitution of amino acids, but retain at least one biological activity. Representative examples of “biological activity” include the ability to bind to a toll-like receptor and to be bound by a specific antibody. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties

Mature human CD24 protein as used herein, is also sometimes referred to as mature human CD24 polypeptide and as human CD24 ECD.

An amino acid modification, as used herein, refers to a substitution of an amino acid, including substitution with any of the 20 amino acids commonly found in human proteins or with an atypical or non-naturally occurring amino acid, or the derivation of an amino acid by the addition and/or removal of chemical groups to/from the amino acid,. Commercial sources of atypical amino acids include Sigma-Aldrich (Milwaukee, Wisc.), ChemPep Inc. (Miami, Fla.), and Genzyme Pharmaceuticals (Cambridge, Mass.). Atypical amino acids may be purchased from commercial suppliers, synthesized de novo, or chemically modified or derivatized from naturally occurring amino acids.

An amino acid substitution, as used herein, refers to the replacement of one amino acid residue by a different amino acid residue (including an atypical or non-naturally occurring amino acid).

A conservative amino acid substitution, as used herein, is defined as exchanges within one of the following five groups of amino acids:

I. Small aliphatic, nonpolar or slightly polar residues: Ala, Ser, Thr, Pro, Gly;

II. Polar, negatively charged residues and their amides: Asp, Asn, Glu, Gln, cysteic acid and homocysteic acid;

III. Polar, positively charged residues: His, Arg, Lys; Ornithine (Orn);

IV. Large, aliphatic, nonpolar residues: Met, Leu, Ile, Val, Cys, Norleucine (Nle), homocysteine;

V. Large, aromatic residues: Phe, Tyr, Trp, acetyl phenylalanine.

### 2. CD24

Provided herein are methods that include administering a CD24 protein. The CD24 protein may comprise a mature CD24 polypeptide or a variant thereof. The mature form of the CD24 protein corresponds to the extracellular domain (ECD). The mature CD24 protein administered as part of the invention may be derived from a human or another mammal. As described above, mature human CD24 protein is 31 amino acids long and has a variable alanine (A) or valine (V) residue at its C-terminal end, as follows:

SETTTGTSSNSSQSTSNSGLAPNPTNATTK(V/A) (SEQ ID NO: 1)

The C-terminal valine or alanine as shown in SEQ ID NO: 1 may be immunogenic and when omitted from the CD24 protein provided herein, may reduce its immunogenicity.

Therefore, the CD24 protein provided herein may comprise the amino acid sequence of mature human CD24 lacking the C-terminal valine or alanine amino acid, as follows:

SETTTGTSSNSSQSTSNSGLAPNPTNATTK (SEQ ID NO: 2)

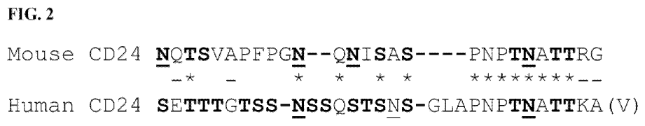

Despite considerable sequence variations in the amino acid sequence of the extracellular domain of mature CD24 proteins from mouse and human, they are functionally equivalent, as a soluble form of human CD24 has been shown to be active in the mouse. The amino acid sequence of the human CD24 (SEQ ID NO: 1) is 39% identical to the corresponding mouse protein (Genbank accession number NP_033976). However, it is not that surprising that the percent identity is not higher as the CD24 is only 27-31 amino acids in length, depending on the species, and binding to some of its receptor(s), such as Siglec 10/G, is mediated by its sialic acid and/or galactose sugars of the glycoprotein. The amino acid sequence identity between the extracellular domains of the human Siglec-10 (GenBank accession number AF310233) and its murine homolog Siglec-G (GenBank accession number NP_766488) receptor proteins is 63% (FIG. 2). Since the sequence conservation between the mouse and human mature CD24 proteins is primarily in the C-terminus of the proteins and there is an abundance of potential glycosylation sites (S and T residues) in the extracellular domain, the CD24 proteins provided herein may tolerate significant variations from the mature CD24 sequence as shown in SEQ ID NO: 1, especially if those variations do not affect the conserved residues in the C-terminus and/or the glycosylation sites. Therefore, the CD24 protein provided herein may comprise the amino acid sequence of mature murine CD24:

NQTSVAPFPGNQNISASPNPTNATTRG (SEQ ID NO: 3).

The amino acid sequence of the human CD24 shows more sequence conservation with the cynomolgus monkey version of the protein (52% identity; UniProt accession number UniProtKB—I7GKK1) than with mouse. Again, it is not surprising given that the percent identity is not higher as the ECD is only 29-31 amino acids in length in these species, and the role of sugar residues in binding to its receptor(s). The amino acid sequence of cynomolgous Siglec-10 receptor has not been determined, but the amino acid sequence identity between the human and rhesus monkey Siglec-10 (GenBank accession number XP 001116352) proteins is 89%. Therefore, the CD24 protein provided herein may also comprise the amino acid sequence of mature cynomolgous (or rhesus) monkey CD24:

TVTTSAPLSSNSPQNTSTTPNPANTTTKA (SEQ ID NO: 10)

The CD24protein administered as part of the disclosed methods may be soluble. The CD24 protein may further comprise an N-terminal signal peptide, to allow secretion from a cell expressing the protein. The signal peptide sequence may comprise the amino acid sequence: MGRAMVARLGLGLLLLALLLPTQIYS (SEQ ID NO: 4). Alternatively, the signal sequence may be any of those that are found on other transmembrane or secreted proteins, or those modified from the existing signal peptides known in the art.

a. Fusion

The CD24 protein administered by the method described herein may be fused at its N-or C-terminal end to a protein tag, which may comprise a portion of a mammalian Ig protein, which may be human or mouse or from another species. The portion may comprise an Fc region of the Ig protein. The Fc region may comprise at least one of the hinge region, CH2, CH3, and CH4 domains of the Ig protein. The Ig protein may be human IgG1, IgG2, IgG3, IgG4, or IgA, and the Fc region may comprise the hinge region, and CH2 and CH3 domains of the Ig. The Fc region may comprise the human immunoglobulin G1 (IgG1) isotype SEQ ID NO: 7. The Ig protein may also be IgM, and the Fc region may comprise the hinge region and CH2, CH3, and CH4 domains of IgM. The protein tag may be an affinity tag that aids in the purification of the protein, and/or a solubility-enhancing tag that enhances the solubility and recovery of functional proteins. The protein tag may also increase the valency of the CD24 protein. The protein tag may also comprise GST, His, FLAG, Myc, MBP, NusA, thioredoxin (TRX), small ubiquitin-like modifier (SUMO), ubiquitin (Ub), albumin, or a Camelid Ig. Methods for making fusion proteins and purifying fusion proteins are well known in the art.

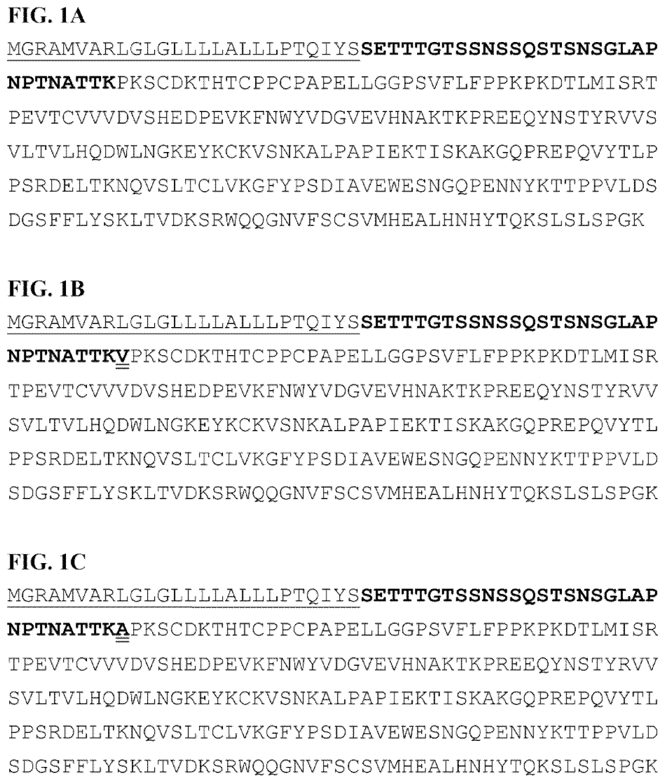

For the construction of the fusion protein CD24Fc identified in the examples, a truncated form of the mature CD24 polypeptide of 30 amino acids as represented by SEQ ID NO: 2, lacking the final (C-terminal) polymorphic amino acid of the mature human CD24 polypeptide (located before the GPI signal cleavage site of the full-length CD24 protein), has been used. This variant of the mature human CD24 polypeptide sequence is fused to a human IgG1 Fc domain represented by SEQ ID NO: 7. A “full-length” version of the CD24Fc fusion protein is provided in SEQ ID NO: 5 (FIG. 1A), which contains the 30 amino acid mature CD24 variant polypeptide and the CD24 signal peptide fused to the IgG1 Fc domain at the C-terminus of the CD24 polypeptide. A processed version of the full-length CD24Fc fusion protein that is secreted from the cell (i.e., lacking the signal sequence, which is cleaved off) is provided in SEQ ID NO: 6, which contains just the 30 amino acid mature CD24 variant polypeptide fused to the IgG1 Fc domain. Processed polymorphic variants of mature CD24 (that is, mature CD24 polypeptide having SEQ ID NO: 1) fused to IgG1 Fc may comprise the amino acid sequence set forth in SEQ ID NO: 11 or 12.

Thus, as used herein, “CD24Fc” is a fusion that corresponds to a variant of the mature human CD24 polypeptide wherein the last (C-terminal) amino acid (A or V) of the mature human CD24 polypeptide has been deleted, and wherein the mature human CD24 polypeptide variant is fused to an IgG1 Fc domain. SEQ ID NO: 6 is an example of such a CD24 fusion protein, wherein the IgG1 Fc domain is fused to the C-terminal of the human mature CD24 polypeptide variant.

b. Production

The CD24 protein administered by the methods described herein, which includes Fc fusion proteins of CD24 as described, may be heavily glycosylated, and may be involved in functions of CD24 protein such as costimulation of immune cells and interaction with a damage-associated molecular pattern molecule (DAMP). The CD24 protein may be prepared using a eukaryotic expression system. The expression system may entail expression from a vector in mammalian cells, such as Chinese Hamster Ovary (CHO) cells. The system may also be a viral vector, such as a replication-defective retroviral vector that may be used to infect eukaryotic cells. The CD24 protein may also be produced from a stable cell line that expresses the CD24 protein from a vector or a portion of a vector that has been integrated into the cellular genome. The stable cell line may express the CD24 protein from an integrated replication-defective retroviral vector. The expression system may be GPEX™ (Catalent Biotechnology, Somerset, N.J.).

c. Pharmaceutical Composition

The CD24 protein administered by the methods described herein, which includes Fc fusion proteins of CD24 as described, may be contained in a pharmaceutical composition, which may comprise a pharmaceutically acceptable amount of the CD24 protein. The pharmaceutical composition may comprise a pharmaceutically acceptable carrier. The pharmaceutical composition may comprise a solvent, which may keep the CD24 protein stable over an extended period. The solvent may be PBS, which may keep the CD24 protein stable for at least 66 months at −20° C. (−15˜−25° C.). The solvent may be capable of accommodating the CD24 protein in combination with another drug.

The pharmaceutical composition may be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water. In one example, the pharmaceutical composition comprises a normal saline solution, in which the CD24 protein may be diluted. The volume of the saline solution may be 100 mL. In one example, the pharmaceutical composition for intravenous administration comprises 480 mg of the CD24 protein, including Fc fusion proteins of mature human CD24 as described.

The pharmaceutical composition may also be formulated as a depot preparation, which may be administered by implantation or by intramuscular injection. The composition may be formulated with suitable polymeric or hydrophobic materials (as an emulsion in an acceptable oil, for example), ion exchange resins, or as sparingly soluble derivatives (as a sparingly soluble salt, for example).

d. Dosage

The dose of the CD24 protein administered by the method described herein, which includes Fc fusion proteins of mature CD24 as described, may ultimately be determined through a clinical trial to determine a dose with acceptable toxicity and clinical efficacy. The initial clinical dose may be estimated through pharmacokinetics and toxicity studies in rodents and non-human primates. The dose of the CD24 protein may be 0.01 mg/kg to 1000 mg/kg, and may be 1 to 500 mg/kg, depending on the desired effect on irAEs or GvHD and the route of administration. The CD24 protein may be administered by intravenous infusion or subcutaneous, intramural (that is, within the wall of a cavity or organ), or intraperitoneal injection, and the dose may be 10-1000 mg, 10-500 mg, 10-240 mg, 10-120 mg, or 10, 30, 60, 120, 240 mg, or 480 mg, where the subject is a human.

### 3. Methods of treatment

a. Pneumonia

Viral pneumonia accounts for more than ⅓ of pneumonia in human and is thus a major cause of mortality. The most frequent cause of viral pneumonia is influenza virus. In addition, parainfluenza and respiratory syncytial virus are frequently causes.

Accordingly, the CD24 protein may be used to treat or prevent a viral pneumonia infection in a subject in need thereof. The pneumonia may be caused by one or more of an influenza virus, a parainfluenza virus, a respiratory syncytial virus, or a human immunodeficiency virus. The pneumonia may be caused by a secondary bacterial infection, which may follow a viral infection. In particular, Staphylococci are the most frequent cause of secondary pneumonia. The CD24 protein used to treat or prevent a viral pneumonia infection resulting from influenza virus, parainfluenza, or respiratory syncytial virus, or a secondary bacterial infection that follows a viral infection, may comprise a mature human CD24 polypeptide or a variant thereof, as illustrated in the sequences set forth in SEQ ID NO: 1 or 2. The CD24 protein used to treat or prevent a viral pneumonia infection resulting from influenza virus, parainfluenza, or respiratory syncytial virus, or a secondary bacterial infection that follows a viral infection, may comprise a protein tag, wherein the protein tag is fused at the N-terminus or C-terminus of the CD24 protein. The protein tag may comprise a portion of a mammalian immunoglobulin (Ig) protein, wherein the portion of the mammalian Ig protein may be a Fc region. The Ig protein may be human. The Fc region may comprise a hinge region and CH₂ and CH₃ domains of a human Ig protein selected from the group consisting of IgG1, IgG2, IgG3, IgG4, and IgA. The Fc region may comprise a hinge region and CH₂, CH₃ and CH₄ domains of IgM. The amino acid sequence of the CD24 protein used to treat or prevent a viral pneumonia infection resulting from influenza virus, parainfluenza, or respiratory syncytial virus, or a secondary bacterial infection that follows a viral infection, may comprise a sequence set forth in SEQ ID NO: 6, 11, or 12. The amino acid sequence of the CD24 protein used to treat or prevent a viral pneumonia infection may comprise a sequence set forth in SEQ ID NO: 6. The amino acid sequence of the CD24 protein used to treat or prevent a viral pneumonia infection resulting from influenza virus, parainfluenza, or respiratory syncytial virus, or a secondary bacterial infection that follows a viral infection, may comprise a sequence set forth in SEQ ID NO: 11. The amino acid sequence of the CD24 protein used to treat or prevent a viral pneumonia infection may comprise a sequence set forth in SEQ ID NO: 12. The amino acid sequence of the CD24 protein used to treat or prevent a viral pneumonia as described herein may also consist of a sequence set forth in SEQ ID NO: 6, 11, or 12.

The CD24 protein used to treat or prevent a viral pneumonia infection resulting from influenza virus, parainfluenza, or respiratory syncytial virus, or a secondary bacterial infection that follows a viral infection, can be produced using a eukaryotic protein expression system wherein the expression system may comprise a vector contained in a Chinese Hamster Ovary cell line or a replication-defective retroviral vector and wherein the replication-defective retroviral vector is stably integrated into the genome of a eukaryotic cell. The CD24 protein used to treat or prevent a viral pneumonia resulting from influenza virus, parainfluenza, or respiratory syncytial virus, or a secondary bacterial infection that follows a viral infection, is soluble and glycosylated. An embodiment includes the use of a CD24 protein as described herein in the manufacture of a medicament for use to treat or prevent a viral pneumonia resulting from influenza virus, parainfluenza, or respiratory syncytial virus, or a secondary bacterial infection that follows a viral infection in a subject.

The subject may be a mammal. In particular, the mammal may be a monkey or an ape. The ape may be a chimpanzee, gorilla, or human.

In both primary and secondary pneumonia, viruses and bacteria may cause death of infected cells, which in turn can trigger inflammation in the lung. Therefore, therapeutics that suppress tissue injury-induced inflammation may also be used to treat viral and bacterial pneumonia.

b. Administration

The route of administration of the CD24 protein pharmaceutical compositions described herein may be parenteral. Parenteral administration includes, but is not limited to, intravenous, intraarterial, intraperitoneal, subcutaneous, intramuscular, intrathecal, intraarticular, and direct injection. The composition may be administered 1, 2, 3, 4, 5, 6, 7, 8, 9, 10, 11, or 12 times per day. The composition may be administered for a period of 1, 2, 3, 4, 5, 6, 7, 8, 9, 10, 11, 12, 13, or 14 days, or 3, 4, 5, 6, 7, 8, 9, 10, 11, or 12 weeks.

In one example, the CD24 protein described herein may be administered at a 480 mg dose. In one embodiment, the CD24 protein may be infused intravenously, which may be administered over a period of up to 30, 40, 50, 60, 70, 80, 90, 100, 110, or120 minutes, or up to 3, 4, 5, 6, 7 or 8 hours. The CD24 protein may be diluted in normal saline for intravenous administration, including for example to a 100 mL volume.

c. Combination Treatment

Since viral and bacterial infections are the primary cause of viral pneumonia, antivirals and antibiotics are frequently used as the treatment of choice. However, since pneumonia are lung inflammation that can be caused by tissue injuries following infection, other treatment can be used in combination with reagents that primarily inhibit infections, such reagents includes commonly used antivirals (for example, remdesivir, or molnupiravir) as well as antibiotics as indicated by specific pathogens. In one example, the subject may be treated with remdesivir in combination with the CD24 protein in the treatment of viral pneumonia. In another example, the subject may be treated with molnupiravir in combination with the CD24 protein in the treatment of viral pneumonia.

The CD24 protein may be administered simultaneously or metronomically with other treatments. The term “simultaneous” or “simultaneously” as used herein, means that the CD24 protein and other treatment be administered within 48 hours, preferably 24 hours, more preferably 12 hours, yet more preferably 6 hours, and most preferably 3 hours or less, of each other. The term “metronomically” as used herein means the administration of the agent at times different from the other treatment and at a certain frequency relative to repeat administration.

The CD24 protein as described herein may be administered at any point prior to another treatment including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50 hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, 1 hr, 55 mins., 50 mins., 45 mins., 40 mins., 35 mins., 30 mins., 25 mins., 20 mins., 15 mins, 10 mins, 9 mins, 8 mins, 7 mins., 6 mins., 5 mins., 4 mins., 3 mins, 2 mins, or 1 mins. The CD24 protein may be administered at any point prior to a second treatment of the CD24 protein including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50 hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, 1 hr, 55 mins., 50 mins., 45 mins., 40 mins., 35 mins., 30 mins., 25 mins., 20 mins., 15 mins., 10 mins., 9 mins., 8 mins., 7 mins., 6 mins., 5 mins., 4 mins., 3 mins, 2 mins, or 1 mins.

The CD24 protein as described herein may be administered at any point after another treatment including about 1 min, 2 mins., 3 mins., 4 mins., 5 mins., 6 mins., 7 mins., 8 mins., 9 mins., 10 mins., 15 mins., 20 mins., 25 mins., 30 mins., 35 mins., 40 mins., 45 mins., 50 mins., 55 mins., 1 hr, 2 hr, 3 hr, 4 hr, 6 hr, 8 hr, 10 hr, 12 hr, 14 hr, 16 hr, 18 hr, 20 hr, 22 hr, 24 hr, 26 hr, 28 hr, 30 hr, 32 hr, 34 hr, 36 hr, 38 hr, 40 hr, 42 hr, 44 hr, 46 hr, 48 hr, 50 hr, 52 hr, 54 hr, 56 hr, 58 hr, 60 hr, 62 hr, 64 hr, 66 hr, 68 hr, 70 hr, 72 hr, 74 hr, 76 hr, 78 hr, 80 hr, 82 hr, 84 hr, 86 hr, 88 hr, 90 hr, 92 hr, 94 hr, 96 hr, 98 hr, 100 hr, 102 hr, 104 hr, 106 hr, 108 hr, 110 hr, 112 hr, 114 hr, 116 hr, 118 hr, or 120 hr. The CD24 protein may be administered at any point prior after a previous CD24 treatment including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50 hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, 1 hr, 55 mins., 50 mins., 45 mins., 40 mins., 35 mins., 30 mins., 25 mins., 20 mins., 15 mins., 10 mins., 9 mins., 8 mins., 7 mins., 6 mins., 5 mins., 4 mins., 3 mins, 2 mins, or 1 mins.

### Example 1

### CD24 Pharmacokinetics in Mice

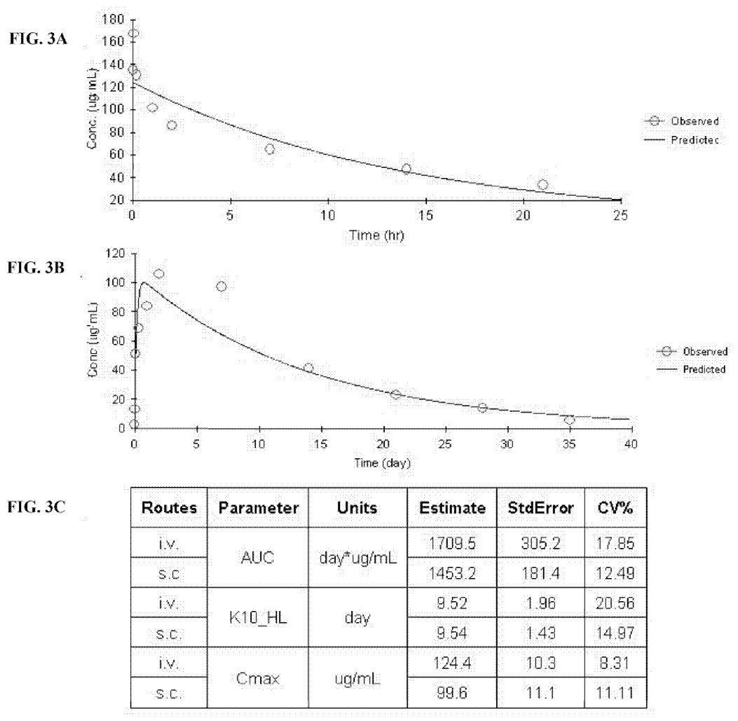

1 mg of CD24Fc represented by SEQ ID NO. 6 was injected into naïve C57BL/6 mice and collected blood samples were collected at different timepoints (5 min, 1 hr, 4 hrs, 24 hrs, 48 hrs, 7 days, 14 days and 21 days) from 3 mice in each timepoint. The sera were diluted 1:100 and the levels of CD24Fc was detected using a sandwich ELISA using purified anti-human CD24 (3.3 μg/ml) as the capturing antibody and peroxidase conjugated goat anti-human IgG Fc (5 μg/ml) as the detecting antibodies. As shown in FIG. 3A. The decay curve of CD24Fc revealed a typical biphasic decay of the protein. The first biodistribution phase had a half-life of 12.4 hours. The second phase follows a model of first-order elimination from the central compartment. The half-life for the second phase was 9.54 days, which is similar to that of antibodies in vivo. These data suggest that the fusion protein is very stable in the blood stream. In another study in which the fusion protein was injected subcutaneously, an almost identical half-life of 9.52 days was observed (FIG. 3B). More importantly, while it took approximately 48 hours for the CD24Fc to reach peak levels in the blood, the total amount of the fusion protein in the blood, as measured by AUC, was substantially the same by either route of injection. Thus, from a therapeutic point of view, using a different route of injection should not affect the therapeutic effect of the drug. This observation greatly simplified the experimental design for primate toxicity and clinical trials.

### Example 2

### CD24-Siglec 10 Interaction in Host Response to Tissue Injuries

Nearly two decades ago, Matzinger proposed what was popularly called danger theory (P. Matzinger, “Tolerance, danger, and the extended family,” Annual Review of Immunology, vol. 12, pp. 991-1045, 1994). In essence, she argued that the immune system is turned on when it senses the dangers in the host. Although the nature of danger was not well defined at the time, it has been determined that necrosis is associated with the release of intracellular components such as HMGB1 and Heat-shock proteins, which were called DAMP, for danger-associated molecular patterns. DAMP were found to promote production of inflammatory cytokines and autoimmune diseases. In animal models, inhibitors of HMGB1 and HSP90 were found to ameliorate rheumatoid arthritis (RA). The involvement of DAMP raised the prospect that negative regulation for host response to DAMP can be explored for RA therapy.

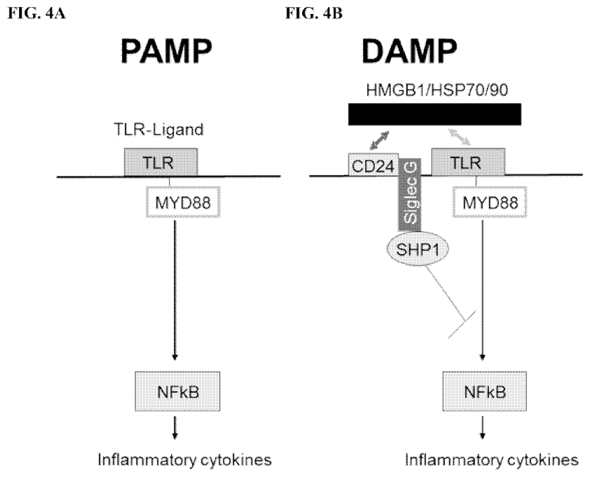

Using acetaminophen-induced liver necrosis and ensuring inflammation, it was observed that through interaction with Siglec G, CD24 provides a powerful negative regulation for host response to tissue injuries. CD24 is a GPI anchored molecules that is broadly expressed in hematopoietic cells and other tissue stem cells. Genetic analysis of a variety of autoimmune disease in human, including multiple sclerosis, systemic lupus erythromatosus, RA, and giant cell arthritis, showed significant association between CD24 polymorphism and risk of autoimmune diseases. Siglec G is a member of I-lectin family, defined by their ability to recognize sialic acid containing structure. Siglec G recognizes sialic acid containing structure on CD24 and negatively regulates production of inflammatory cytokines by dendritic cells (DC). In terms of its ability to interact with CD24, human Siglec 10 and mouse Siglec G are functionally equivalent. However, it is unclear if there is a one-to-one correlation between mouse and human homologues. Although the mechanism remains to be fully elucidated, it is plausible that SiglecG-associated SHP1 may be involved in the negative regulation. These data lead to a new model in which CD24-Siglec G/10 interaction may play a critical role in discrimination pathogen-associated molecular pattern (PAMP) from DAMP (FIG. 4).

At least two overlapping mechanisms may explain the function of CD24. First, by binding to a variety of DAMP, CD24 may trap the inflammatory stimuli to prevent their interaction with TLR or RAGE. This notion is supported by observations that CD24 is associated with several DAMP molecules, including HSP70, 90, HMGB1 and nucleolin. Second, perhaps after associated with DAMP, CD24 may stimulate signaling by Siglec G. Both mechanisms may act in concert as mice with targeted mutation of either gene mounted much stronger inflammatory response. In fact, DC cultured from bone marrow from either CD24−/− or Siglec G−/− mice produced much higher inflammatory cytokines when stimulated with either HMGB1, HSP70, or HSP90. In contrast, no effect were found in their response to PAMP, such as LPS and PolyI:C. These data not only provided a mechanism for the innate immune system to distinguish pathogen from tissue injury, but also suggest CD24 and Siglec G as potential therapeutic targets for diseases associated with tissue injuries. Although CD24-Siglec interaction does not control infection, it can also affect inflammatory response during viral and bacterial infection. For instance, many infections cause tissue injury. In addition, many infectious agents may disrupt CD24-Siglec interaction, as they encode sialidase that removes sialic acid from CD24 and thereby inactivate the negative regulation of inflammation caused by tissue injuries.

### Example 3

CD24Fc Interacts with HMGB1, Siglec 10 and Induces Association between Siglec G and SHP-1

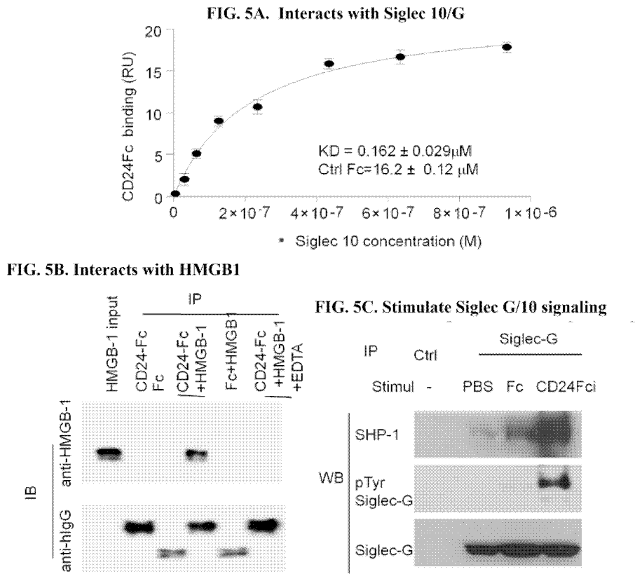

To measure the interaction between CD24Fc and Siglec 10, we immobilized CD24Fc onto a CHIP and used Biacore to measure the binding of different concentrations of Siglec-10Fc. As shown in FIG. 5A, CD24Fc binds with Siglec 10 with a Kd of 1.6×10⁻⁷M. This is 100-fold higher affinity than the control Fc. The interaction between CD24Fc and HMGB1 was confirmed by pull down experiments using CD24Fc-bound protein G beads followed by Western blot with either anti-IgG or anti-HMGB1. These data demonstrate that CD24Fc, but not Fc, binds to HMGB1 and that this binding is cation-dependent (FIG. 5B). To determine whether CD24Fc is an agonist of Siglec G, the mouse counterpart of human Siglec 10, we stimulated CD24−/− spleen cells with CD24Fc, control Fc or vehicle (PBS) control for 30 minutes. Siglec G was then immunoprecipitated and probed with anti-phospho-tyrosine or anti-SHP-1. As shown in FIG. 5C, CD24Fc induced substantial phosphorylation of Siglec G and association of SHP-1, a well-known inhibitor for both adaptive and innate immunity.

In vitro efficacy studies of CD24Fc.

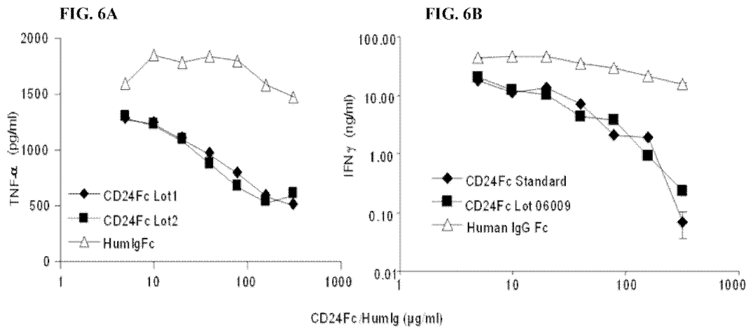

To study the impact of CD24Fc on the production of inflammatory cytokines by human T cells, the mature T cells in human PBML were activated by anti-CD3 antibody (OKT3), a commonly used agonist of the T cell receptor in the presence of different concentrations of CD24Fc or human IgG1 Fc. Four days later, the supernatants were collected and the production of IFN-γ and TNF-α were measured by Enzyme-linked immunosorbent assay (ELISA) to confirm activation. The results in FIG. 6 demonstrated that CD24Fc from two different manufacturing lots significantly reduced IFN-γ and TNF-α production from the activated human PBML compared with control IgG Fc control. In addition, when CD24Fc was added, cytokine production was inhibited in a dose-dependent manner. Therefore, CD24Fc can inhibit anti-CD3 induced human PBML activation in vitro. This study not only indicated the mechanism of action of CD24Fc might be through the inhibition of T cell activation, but also established a reliable bioassay for drug potency and stability testing.

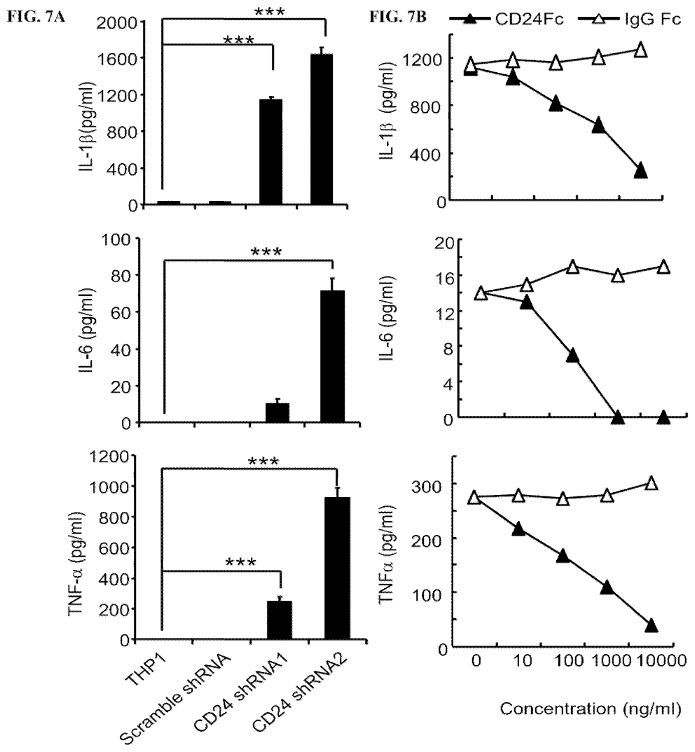

To determine whether CD24Fc regulates production of inflammatory cytokines in a human cell line, we first silenced CD24 in the human acute monocytic leukemia THP1 cell line using RNAi, and then induced differentiation into macrophages by treating them with PMA. As shown in FIG. 7A, CD24 silencing substantially increased the production of TNFα, IL-1β and IL-6. These data demonstrate an essential role for endogenous human CD24 in limiting the production of inflammatory cytokines. Importantly, CD24Fc restored inhibition of TNFα in the CD24-silenced cell line (FIG. 7B), as well as IL-1β and IL-6. These data not only demonstrate the relevance of CD24 in inflammatory response of human cells, but also provides a simple assay to assess biological activity of CD24Fc.

Taken together, these data demonstrate that CD24Fc is capable of inhibiting cytokine production triggered by adaptive and innate stimuli. However, since the drug is much more effective in reducing cytokine production by innate effectors, we consider that the primary mechanism for its prophylactic function is to prevent inflammation triggered by tissue injuries at the early phase of transplantation.

### Example 4

### CD24 and the Treatment or Prevention of Viral Pneumonia

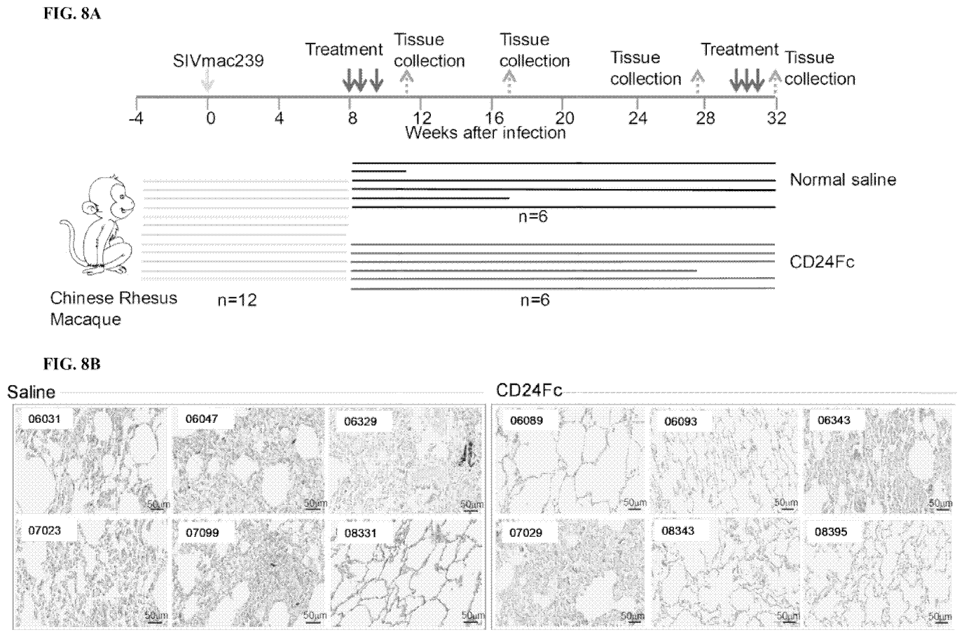

This example demonstrates that a CD24 protein described herein can be used to treat or prevent a viral pneumonia. This was accomplished by using a Simian immunodeficiency virus (SIV) model, in which infected animals develop pneumonia. As diagrammed in FIG. 8A, 12 Chinese rhesus macaques were infected with SIVmac239 via intravenous infusion of 4000 50% tissue culture infective doses of SIVmac239, and randomized into test and control groups, respectively receiving 3 injections of CD24Fc or normal saline (NS) on day 56 of infection. Five months later, another cycle of treatments was given to surviving monkeys, which were terminated one week after the last dosing for biomarker studies. Lung sections from all randomized animals, regardless of whether they completed all dosing, were included for analysis, as detailed in Table 1 below.

TABLE 1
Summary of SIV-infected animal information
after treatment with CD24Fc or Control

| | | Cycle 1 | Cycle 2 | Tissue | |
|---|---|---|---|---|---|
| Animal | Treat- | 56-66 | 176-186 | collection | Symptom/diagnosis/ |
| ID | ment | dpi | dpi | dpi | pathological findings |
| 06031 | PBS | + | + | 190 | Intractable diarrhea, |
| | | | | | wasting syndrome/ |
| | | | | | AIDS/pneumonia |
| 06047 | PBS | + | − | 80 | wasting syndrome/ |
| | | | | | death/pneumonia |
| 06329 | PBS | + | + | 190 | AIDS/pneumonitis/ |
| | | | | | wasting syndrome |
| 07023 | PBS | + | + | 190 | AIDS/pneumonia |
| 07099 | PBS | + | − | 119 | Intractable diarrhea, |
| | | | | | wasting syndrome/ |
| | | | | | death/pneumonia |
| 08331 | PBS | + | + | 190 | Intractable diarrhea/ |
| | | | | | wasting syndrome/ |
| | | | | | AIDS/normal |
| 06089 | CD24Fc | + | + | 190 | healthy/normal |
| 06093 | CD24Fc | + | + | 190 | AIDS/normal/wasting |
| | | | | | syndrome |
| 06343 | CD24Fc | + | + | 190 | healthy/pneumonia |
| 07029 | CD24Fc | + | − | 174 | death/wasting |
| | | | | | syndrome/pneumonia |
| 08343 | CD24Fc | + | + | 190 | healthy/normal |
| 08365 | CD24Fc | + | + | 190 | healthy/normal |

The lung sections from either necropsy or autopsy were fixed with 4% paraformaldehyde and stained with hematoxylin and eosin (H&E). H&E sections were blinded and independently scored by two researchers for viral pneumonia. Blinded scoring revealed that 5 or 6 (83%) control animals developed severe pneumonia, while 2 or 6 CD24Fc-treated animals developed severe pneumonia, representing a substantial reduction of pneumonia. The most prominent finding shared in both groups of monkeys was interstitial pneumonia (06031, 06047, 06329, and 07099 in NS group; 06343 and 07029 in CD24Fc group).

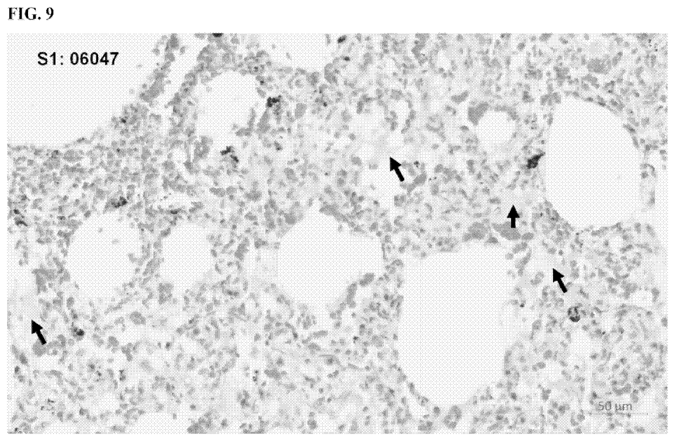

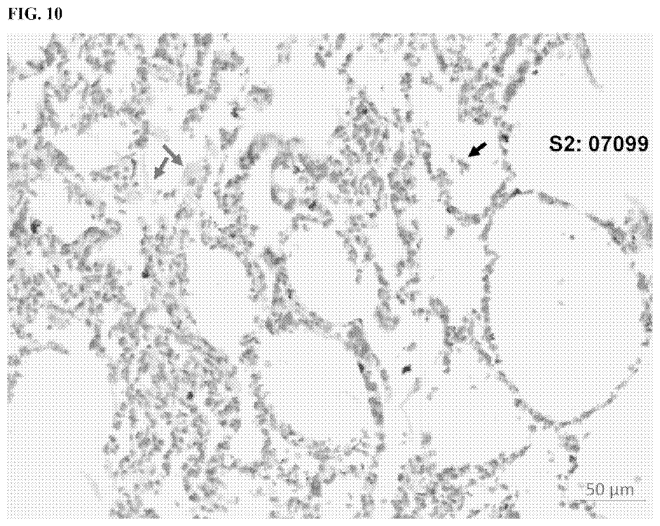

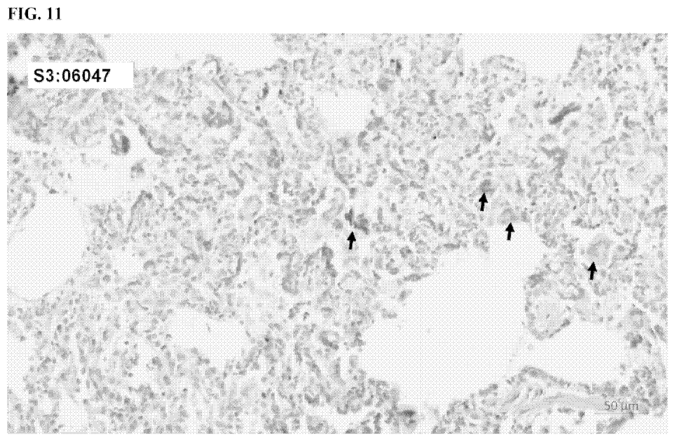

In addition to a reduction in the rate of pneumonia, substantial difference was observed in pathology features between control and the CD24Fc treated groups. Two control monkeys died within 9 weeks after completion of first cycle of treatment with PBS(Table 1). Both monkeys had pathology features of acute respiratory distress syndrome (ARDS), including hyaline membranes lining alveolar walls in monkey No. 06047 (FIG. 9) and alveolar hyaline formation and desquamation of pneumocytes in monkey No. 07099 (FIG. 10). Since these features were not observed in the CD24Fc group, these data suggest CD24Fc may have protected monkeys against ARDS. Other features found in the control but not CD24Fc-treated group include hemorrhage (06031, 06047 and 07023), giant cell formation (06047 in control group, FIG. 11) and perivascular inflammation (06329 and 7099 in control group).

Taken together, the data revealed that CD24Fc not only reduced incidence of viral pneumonia but also qualitatively altered the nature of immunopathology in the lung. Previous studies have demonstrated that lung lesions developed within 2-4 weeks of SIV-infected rhesus monkey (Baskin, G. B. et al. Lentivirus-induced pulmonary lesions in rhesus monkeys (Macacamulatta) infected with simian immunodeficiency virus, Vet Pathol, Vol. 28, pp. 506-513 (1991)). By 8 weeks, essentially all monkeys have developed lung pathology, including perivascular inflammation, vasculitis and interstitial pneumonia and syncytial cells. Since the CD24 treatment was initiated at 8 weeks after infection, the data demonstrate that CD24Fc has therapeutic effects for SIV-induced lung inflammatory lesions, and that a CD24 protein described herein may be used to treat or prevent viral pneumonia.

1. A method of treating or preventing a viral pneumonia in a subject in need thereof, comprising administering a CD24 protein to the subject.

2. The method of claim 1, wherein the pneumonia is caused by an influenza virus, a parainfluenza virus, a respiratory syncytial virus, or a human immunodeficiency virus.

3. The method of claim 1, wherein the pneumonia is caused by a secondary bacterial infection following a viral infection.

4. The method of claim 1, wherein the CD24 protein comprises a mature human CD24 polypeptide or a variant thereof.

5. The method of claim 4, wherein the mature human CD24 polypeptide or variant thereof comprises a sequence set forth in SEQ ID NO: 1 or 2.

6. The method of claim 4, wherein the mature human CD24 protein comprises a protein tag, wherein the protein tag is fused at the N-terminus or C-terminus of the CD24 protein.

7. The method of claim 6, wherein the protein tag comprises a portion of a mammalian immunoglobulin (Ig) protein.

8. The method of claim 7, wherein the portion of the mammalian Ig protein is a Fc region.

9. The method of claim 8, wherein the Ig protein is human.

10. The method of claim 9, wherein the Fc region comprises a hinge region and CH2 and CH3 domains of a human Ig protein selected from the group consisting of IgG1, IgG2, IgG3, IgG4, and IgA.

11. The method of claim 9, wherein the Fc region comprises a hinge region and CH2, CH3 and CH4 domains of IgM.

12. The method of claim 1, wherein the amino acid sequence of the CD24 protein comprises a sequence set forth in SEQ ID NO: 6, 11, or 12.

13. The method of claim 12, wherein the amino acid sequence of the CD24 protein consists of the sequence set forth in SEQ ID NO: 6, 11, or 12.

14. The method of claim 1, wherein the CD24 protein is produced using a eukaryotic protein expression system.

15. The method of claim 14, wherein the expression system comprises a vector contained in a Chinese Hamster Ovary cell line or a replication-defective retroviral vector.

16. The method of claim 15, wherein the replication-defective retroviral vector is stably integrated into the genome of a eukaryotic cell.

17. The method of claim 1, wherein the CD24 protein is soluble.

18. The method of claim 1, wherein the CD24 protein is glycosylated.

19-23. (canceled)

24. The method of claim 5, wherein the mature human CD24 polypeptide comprises a sequence set forth in SEQ ID NO: 1.

25. The method of claim 5,wherein the mature human CD24 polypeptide comprises a sequence set forth in SEQ ID NO: 2.

26-41. (canceled)
